(19) United States
(12) Patent Application Publication
(10) Pub. No.: US 2024/0319201 A1
(43) Pub. Date: Sep. 26, 2024

(54) Systems and Methods for Digital, Multiplexed, Extracellular Vesicle-Derived Biomarker Diagnostic Lab-on-a-Chip

(71) Applicant: Bryan Joseph Rice, Hillsboro, OR (US)

(72) Inventor: Bryan Joseph Rice, Hillsboro, OR (US)

(21) Appl. No.: 18/438,333

(22) Filed: Feb. 9, 2024

Related U.S. Application Data
(60) Provisional application No. 63/444,986, filed on Feb. 12, 2023.

(51) Int. Cl. G01N 33/58; G01N 27/447; G01N 27/49

(52) U.S. Cl. CPC G01N 33/587; G01N 27/4473; G01N 27/44791; G01N 27/49; G01N 33/58

(57) ABSTRACT

This disclosure relates generally to lab-on-chip diagnostic platforms, and in particular, relates to detection of extracellular vesicle biomarkers using lab-on-a-chip diagnostics. The properties of extracellular vesicles provide the opportunity for early detection of biomarkers corresponding to early disease. Combinatorial detection of the presence of multiple cancer-associated biomarkers from extracellular vesicles along with analysis with advanced machine learning algorithms, may be useful for sensitive and specific diagnosis of early cancer and other diseases from biological fluids. Disclosed herein are compositions, methods, and exosome detection apparatus for biomarker detection.

### CROSS REFERENCE TO RELATED APPLICATIONS

This application claims the benefit of U.S. Provisional Application Ser. No. 63/444,986 filed Feb. 12, 2023, the contents of which are incorporated herein by reference.

### TECHNICAL FIELD

This disclosure relates generally to lab-on-chip diagnostic platforms, and in particular, relates to detection of extracellular vesicle biomarkers using lab-on-a-chip diagnostics.

### BACKGROUND

There is a growing demand for diagnostic markers for early disease detection, which can increase the odds of survivability. Early disease detection requires sensitive diagnostic tools to detect low quantities of biomarkers indicative of disease. Few methods exist with the sensitivity and specificity necessary to detect diseases such as cancer and neurodegenerative diseases before considerable progression has taken place. Thus, it may be useful to develop highly sensitive lab-on-chip tests targeting the detection of early disease.

Extracellular vesicles (EVs) are membranous nanoparticles that facilitate intercellular communication system via their biomolecular components (e.g., proteins, lipids, carbohydrates, and nucleic acids). EVs are dense information compartments continuously released from originating cells which contain biomarkers that mimic those of their originating cells. EVs are present in biological fluids (e.g., blood, urine, cerebrospinal fluid, etc.), and EV-associated markers can exhibit longer half-lives and increased stability than free circulating biomarkers. Thus, EVs provide an accessible source of biomarkers that are continuously released from live cells within the body.

Detection methods for cancers and neurodegenerative diseases rely upon costly, time intensive, and often invasive methods (e.g., tissue biopsy, computerized tomography, magnetic resonance imaging, endoscopy, etc.), and many of these diseases have no available tests for early detection. Currently, liquid biopsy tests rely on free circulating markers released during tumor cell death, rather than a continuous and sustained cellular process such as EV secretion. Thus, EVs represent a valuable bio-compartment for the early, minimally-invasive detection of disease-associated biomarkers from their parent cells (e.g., tumor cells, neurons affected by neurodegeneration, inflammatory cells, etc.). Existing EV-based detection methods use either dirty, “brute force” concentration methods like centrifugation and size exclusion or rely upon dilute circulating concentrations. Many existing EV marker test methods use fluorescence-based detection, which is limited in sensitivity and specificity. It may therefore be useful to improve upon the state of the art via efficient biomarker concentration and sensitive, specific biomarker detection using an electronic method.

### BRIEF DESCRIPTION OF THE DRAWINGS

FIG. 1A illustrates an example diagram of example surface chemistry and capture approaches.

FIG. 1B illustrates an example diagram of example surface chemistry and capture approaches.

FIG. 1C illustrates an example diagram of example surface chemistry and capture approaches.

FIG. 1D illustrates an example diagram of example surface chemistry and capture approaches.

FIG. 1E illustrates an example diagram of example surface chemistry and capture approaches.

FIG. 2 illustrates an example flowchart of sample preparation and isolation and/or concentration chambers.

FIG. 3 illustrates an example diagram of a cross section view of a dielectrophoresis concept.

FIG. 4 illustrates an example diagram of a dielectrophoresis electrode array.

FIG. 5A illustrates an example isometric diagram of a generalized fluidics delivery approach.

FIG. 5B illustrates an example isometric diagram of a generalized fluidics delivery approach.

FIG. 6 illustrates an example diagram of device architecture for interrogating multiple EV-derived biomarkers from one or more biological samples using fluidic multiplexing.

FIG. 7 illustrates an example diagram of device architecture for interrogating multiple EV-derived biomarkers from one or more biological samples using fluidic multiplexing.

FIG. 8 illustrates an example diagram of device architecture for interrogating multiple EV-derived biomarkers from one or more biological samples using fluidic multiplexing.

FIG. 9 illustrates an example schematic diagram displaying open circuit potential of a working electrode.

FIG. 10 illustrates an example schematic diagram displaying an example electrode configuration.

FIG. 11 illustrates an example diagram of an electrode configuration in which one embodiment may operate.

FIG. 12 illustrates an example schematic diagram displaying an example electrode configuration.

FIG. 13 illustrates an example diagram of an electrode configuration in which one embodiment may operate.

FIG. 14 illustrates a diagram of an example computer system.

### DESCRIPTION OF EXAMPLE EMBODIMENTS

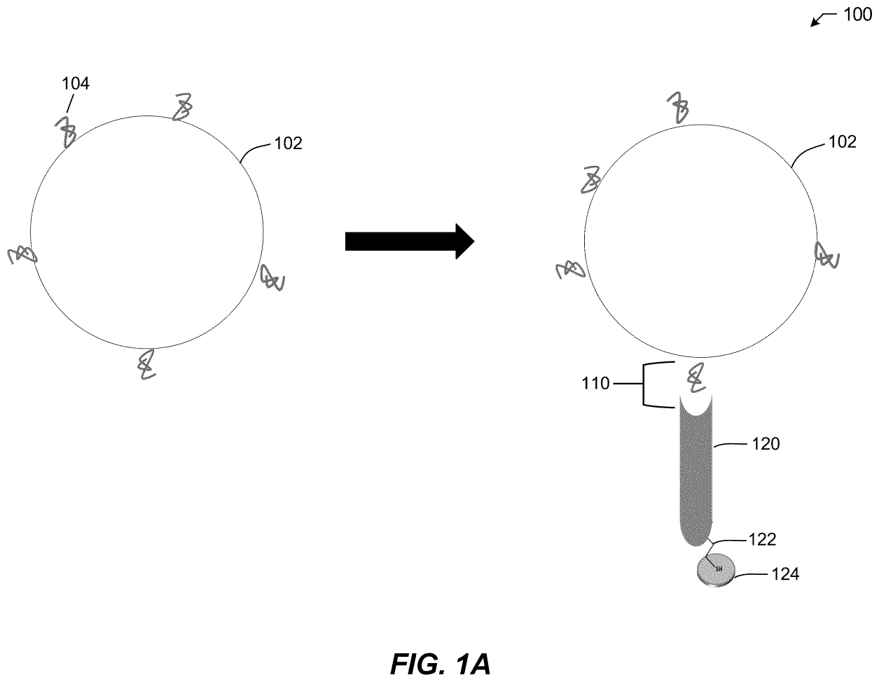

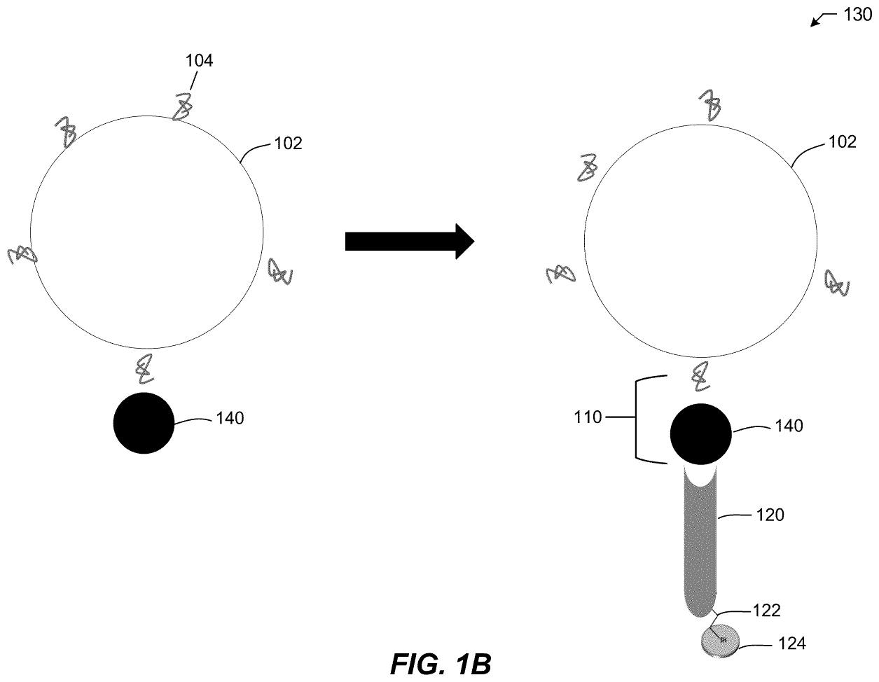

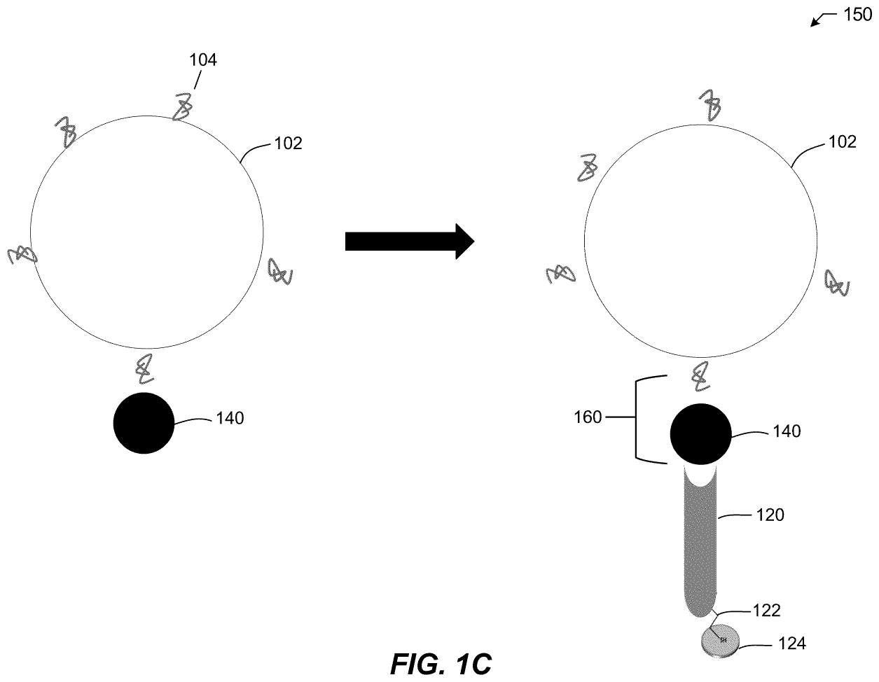

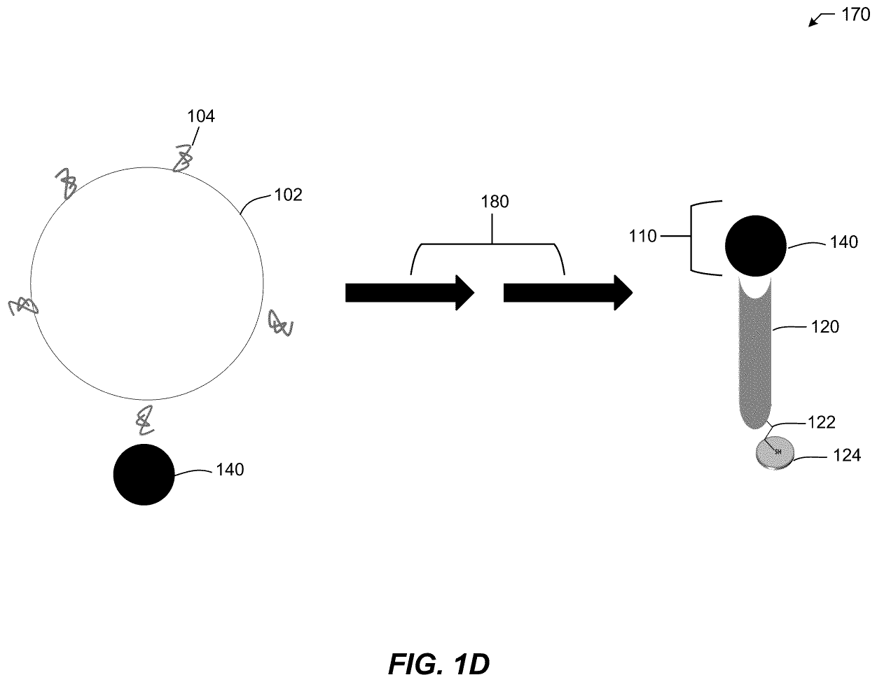

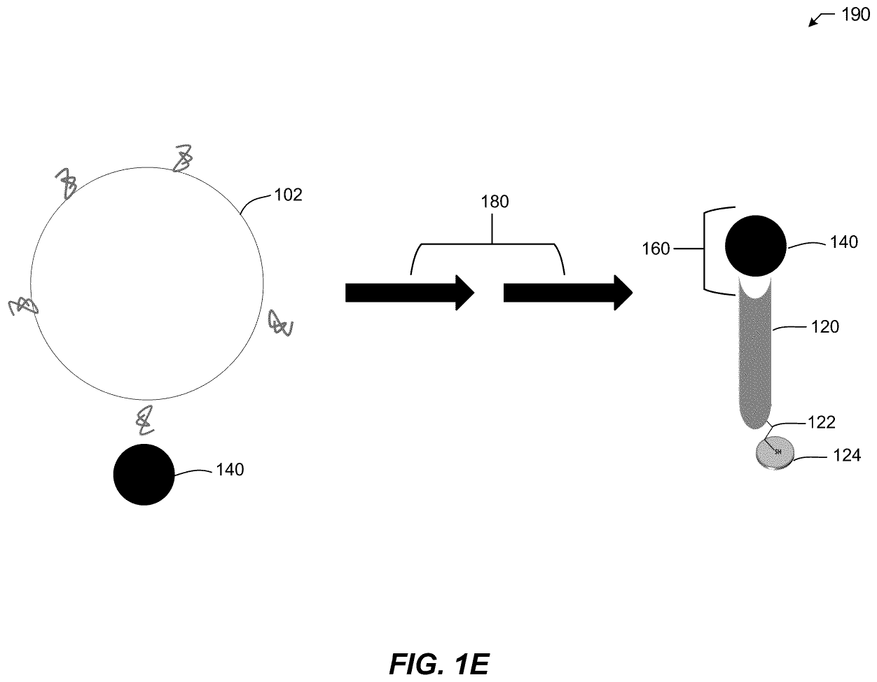

FIG. 1A illustrates an example diagram 100 of example surface chemistry and capture approaches. As used herein, “extracellular vesicles (EVs)” may refer to membranous nanoparticles secreted from cells that can facilitate intercellular communication via their biomolecular components. In particular embodiments, one or more EVs 102 may contain biomarkers 104 on the surface of the EV 102. The previously discussed properties of EVs 102 may provide the opportunity for early detection of biomarkers corresponding to early disease. Thus, combinatorial detection of the presence of multiple cancer-associated biomarkers from EVs 102, along with analysis with advanced machine learning (ML) algorithms, may be useful for sensitive and specific diagnosis of early cancer and other diseases from biological fluids. Disclosed herein are compositions, methods, and exosome detection apparatus for biomarker detection.

In particular embodiments, a biological sample may be loaded onto an electrode array, wherein EVs 102 may be concentrated and/or isolated using dielectrophoresis (DEP). The detection apparatus (e.g., lab-on-a-chip, device) may utilize integral microfluidics (either conventional/pneumatic and/or digital) to divide the concentrated EV volume and automate a multi-marker antibody-based capture. As an example, and not by way of limitation, the respective biomarker concentrations may be directly quantified using an electro-chemical sensor, wherein data from the sensor may be processed through one or more algorithms, resulting in a composite result.

In particular embodiments, biological fluids may be collected via standard point of care procedures, including but not limited to vacutainer tube-based blood draw (e.g., in the case of blood), spinal tap (e.g., in the case of cerebrospinal fluid), and urine collection devices (e.g., in the case of urine). In particular embodiments, biological fluids may be processed to remove interfering cells. As an example, and not by way of limitation, processing of the biological fluids may involve centrifugation, membrane-based filtration, and/or other standard preparation procedures to prepare biological samples for testing. The resulting samples may be plasma, serum, CSF, and/or urine.

In particular embodiments, the prepared biological sample may be flowed over an array of energized electrodes, which impart a dielectrophoretic (DEP) force on particles within the biological sample. The strength and direction of the DEP force may be specific to a plurality of biological particles within the sample, allowing EVs 102 to be isolated from the biological sample and further concentrated. As used herein, “dielectrophoresis (DEP)” may refer to a phenomenon in which a force is exerted on a dielectric particle, molecule, or macromolecular structure in an aqueous or organic solution when it is subjected to a non-uniform electric field.

In particular embodiments, the isolated and concentrated EVs may be mixed with immunochemical reagents and allowed to incubate. After immunochemistry, the respective one or more samples may be loaded onto a sensor array. As an example, and not by way of limitation, the original volume of the EVs 102 may or may not be split into multiple droplets at any point during this process, depending on the necessary workflow.

In particular embodiments, one or more EVs 102 may be floating within a solution, wherein biomarkers 104 may be found on the surface of EV 102. As an example, and not by way of limitation, labeling of biomarkers 104 may include but is not limited to binding of an antibody, an antibody coupled to an enzyme, an antibody coupled to a metal nanoparticle, an antibody coupled to a cleavable single-stranded DNA barcode, an antibody coupled to a cleavable single-stranded DNA barcode that is coupled to a metal nanoparticle, an antibody coupled to a cleavable single-stranded DNA barcode that is coupled to an enzyme, an antibody coupled to a cleavable single-stranded DNA barcode that is coupled to an enzyme, and/or an antibody coupled to a cleavable single-stranded DNA barcode that is coupled to an enzyme. The cleavable linkages to the antibody could include but are not limited to proteolytic, chemical, electrochemical, and photolytic labile compounds. As an example, and not by way of limitation, EV 102 may contain particular biomarkers of interest on the surface of EV 102, wherein the detector surface may be functionalized to capture EV 102 as well as biomarker 104, wherein the detectable event may be used to quantify the biomarker's presence. Table 1, below, outlines a plurality of configurations, EV labels, label release mechanisms, surface capture mechanisms, and detected events.

TABLE 1
Example Configurations of EV labels, Label Release Mechanisms,
Surface Capture Mechanisms, and Detected Events

| | | Label Release | Surface Capture | |
|---|---|---|---|---|
| Config. | EV label | Mechanism | Mechanism | Event Detected |
| A | None | None | Biomarker-specific | Antibody- |
| | | | antibody | biomarker |
| | | | | binding |
| B | Antibody | None | Secondary (capture) | Antibody- |
| | | | antibody binding to EV | antibody |
| | | | label antibody | binding |
| C | Antibody- | None | Secondary (capture) | Enzymatic |
| | enzyme | | antibody binding to EV | activity |
| | | | label antibody | |
| D | Antibody-MNP | None | Secondary (capture) | MNP proximity |
| | | | antibody binding to EV | |
| | | | label antibody | |
| E-1 | Antibody- | Chemical | DNA barcode hybridizing | DNA |
| | ssDNA barcode | | to complementary ssDNA | hybridization |
| E-2 | Antibody- | Photo | DNA barcode hybridizing | DNA |
| | ssDNA barcode | | to complementary ssDNA | hybridization |
| E-3 | Antibody- | Electro- | DNA barcode hybridizing | DNA |
| | ssDNA barcode | chemical | to complementary ssDNA | hybridization |
| F-1 | Antibody- | Chemical | DNA barcode hybridizing | Polymerase |
| | ssDNA barcode | | to complementary ssDNA | activity on |
| | | | | dsDNA |
| F-2 | Antibody- | Photo | DNA barcode hybridizing | Polymerase |
| | ssDNA barcode | | to complementary ssDNA | activity on |
| | | | | dsDNA |
| F-3 | Antibody- | Electro- | DNA barcode hybridizing | Polymerase |
| | ssDNA barcode | chemical | to complementary ssDNA | activity on |
| | | | | dsDNA |
| G-1 | Antibody- | Chemical | DNA barcode hybridizing | MNP proximity |
| | ssDNA barcode- | | to complementary ssDNA | |
| | MNP | | | |
| G-2 | Antibody- | Photo | DNA barcode hybridizing | MNP proximity |
| | ssDNA barcode- | | to complementary ssDNA | |
| | MNP | | | |
| G-3 | Antibody- | Electro- | DNA barcode hybridizing | MNP proximity |
| | ssDNA barcode- | chemical | to complementary ssDNA | |
| | MNP | | | |
| H-1 | Antibody- | Chemical | DNA barcode hybridizing | Enzyme activity |
| | ssDNA barcode- | | to complementary ssDNA | |
| | enzyme | | | |
| H-2 | Antibody- | Photo | DNA barcode hybridizing | Enzyme activity |
| | ssDNA barcode- | | to complementary ssDNA | |
| | enzyme | | | |
| H-3 | Antibody- | Electro- | DNA barcode hybridizing | Enzyme activity |
| | ssDNA barcode- | chemical | to complementary ssDNA | |
| | enzyme | | | |

As displayed in Table 1, “ssDNA” may refer to single-stranded DNA, and “MNP” may refer to a metal nanoparticle. As demonstrated in FIG. 1A, diagram 100 represents configuration “A”, as shown in Table 1, wherein there is no EV label or label release mechanism, the surface capture mechanism is a biomarker-specific antibody, and the event detected by binding event 110 is antibody-biomarker binding.

In particular embodiments, a binding event 110 may be detected between biomarker 104 of EV 102 and capture molecule 120 by a sensor. As an example, and not by way of limitation, capture molecule 120 may be captured by an antibody. As used herein “binding event” 110 may refer to a detectable event used to quantify the presence of a particular biomarker 104. As an example, and not by way of limitation, when binding event 110 occurs, the observed event may either change the capacitance of surface chemistry 122 or increase the number of charge carriers, either of which event may be electronically measured. As particular biomarkers 104 may vary in sensitivity, the detection apparatus may be tuned to a particular range of interest.

As displayed in diagram 100 of FIG. 1A, capture molecule 120 may be an antibody. As an example and not by way of limitation, a method of capturing EV-derived biomarkers (e.g., biomarkers 104) and/or EV-derived biomarker labels may include exposing the electrode surface 124 linked to the antibody 120 (e.g., capture molecule) directly to EVs 102 with biomarker 104, such that biomarker 104, attached to EV 102, directly binds and is captured by antibody 120.

In particular embodiments, a method of capturing EV-derived biomarkers and/or EV-derived biomarker labels may comprise exploiting the chemical reactivity of the surface chemistry 122 to covalently attach antibodies to the surface, wherein the antibody 120 may directly bind its EV-derived biomarker 104.

In particular embodiments, one or more surfaces 124 of field effect transistors may be functionalized using standard surface chemistry processes, such that chemical functional groups may be physically and/or chemically bound through covalent bonds to metals, metal oxides, glassy carbon, graphene, graphene nanoribbons, carbon nanotubes, semiconductors, and/or dielectric surfaces. As used herein “surface chemistry” 122 may refer to the physical and chemical phenomena that occur at the interface between two phases. Surface chemistry 122 may include a molecule with multiple chemical and/or physical sites of reactivity, whereby one site may interact physically and/or chemically with the surface, and the other site is used to directly conjugate nucleic acids, proteins, and/or other molecules of interest. An example of surface chemistry 122 might include but not limited to molecules, nanoparticles and/or biomolecules containing functional groups such as silanes, thiols, disulfides, phosphonates, phosphonic acids, diazonium, alkenes, carboxylic acids, alkynes, alkanes, amines, ketones, esters, aldehydes, alcohols, amides, imines, hydrazines, ethers, nitriles, aromatics, halides and azides. As an example, and not by way of limitation, surface chemistry 122 may be directly conjugated to nucleic acids, proteins, glycans, lipids, and/or other molecules used in the capture and/or detection of biomarkers of interest. In particular embodiments, an insulated-gate field-effect (IGFET) structure may functionalize the gate oxide rather than a metal gate.

In particular embodiments, a method for manufacturing one or more electrodes to detect one or more EV-derived biomarkers may include functionalizing a metal and/or metal oxide surface (hereinafter “surface”) with one or more chemical groups and herein may be referenced as surface chemistry 122. As an example, and not by way of limitation, the chemical reactivity of the surface may be exploited to covalently attach single-stranded DNA to the surface. As another example and not by way of limitation, the inherent surface reactivity may be exploited to attach functionalized single-stranded DNA directly to the surface.

In particular embodiments, the working electrode surface 124 may function as a detector of one or more biomarkers 104. As used herein, the “working electrode surface” may refer to one or more electrodes in an electrochemical system on which the reaction of interest is occurring. In particular embodiments, one or more EVs 102 may be subsequently brought within close proximity of the metal gates of one or more working electrode surfaces 124 during biomarker 104 capture and/or detection mediated by the biomolecules and/or molecules conjugated to the gate surface (e.g . . . , DNA-based hybridization, antibody binding, and/or enzymatic reactions). As an example, and not by way of limitation, the one or more EVs 102 may be concentrated by DEP and brought within close proximity of the metal gates of one or more working electrode surfaces 124 (e.g., capture molecule 120). In particular embodiments, one or more EVs 102 within the biological sample may be labeled via one or more methods, wherein the labeled EV may be attached to the surface of working electrode surface 124. In particular embodiments, the label on one or more EVs 102 may be cleaved from the respective biomarker 104, wherein working electrode surface 124 may capture only the label sans EV 102 at the surface via surface chemistry 122. As an example, and not by way of limitation, a method of releasing labels that are specific for EV-derived biomarkers may be comprised of a linker that can be cleaved chemically, photolytically, and/or electrochemically. Linkers between biomarker 104 and the detection biomolecule and/or molecule that could be cleaved chemically may include, but are not limited to, esters, carbamates, dialkoxydiphenylsilanes azos, diazos, acylhydrazones, nitrobenzenesulfonamides, acylsulfoamides, and/or disulfides. Linkers between biomarker 104 and the detection biomolecule and/or molecule that could be cleaved photolytically may include, but are not limited to, nitrophenyl ethyl ethers and/or phenacyl esters. Linkers between biomarker 104 and the detection biomolecule and/or molecule that could be cleaved electrochemically may include, but are not limited to, aryl esters and imines.

In particular embodiments, digital sensing may occur when EV biomarkers of interest 104 are detected by changes in ion concentration near the gate surface of working electrode surface 124, mediated by, for example, antibody binding events, nucleic acid hybridization events, and/or enzymatic reactions. As an example, and not by way of limitation, the interaction between a biomarker 104 and its capture antibody 120 on the conjugated gate surface modulates the electrical properties of the gate of the field effect transistor, resulting in changes to either the voltage or current across the transistor, depending on the surrounding circuit configuration. This electrical signal may then be amplified and digitized using an analog-to-digital converter.

In particular embodiments, digital output for one or more biomarkers 104 may be analyzed by one or more machine-learning algorithms. As an example and not by way of limitation, the one or more machine-learning algorithms may include supervised, unsupervised, semi-supervised, deep, and/or reinforcement learning algorithms. In particular embodiments, the deep learning algorithms may include any artificial neural networks (ANNs) that may be utilized to learn deep levels of representations and abstractions from large amounts of data. For example, the deep learning algorithms may include ANNs, such as a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), deep reinforcement learning, and so forth.

In particular embodiments, digital output for one or more biomarkers 104 may be analyzed by one or more classification machine-learning algorithms or functions which may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon.

Although this disclosure references the aforementioned machine-learning algorithms, this disclosure contemplates any suitable machine-learning algorithm. In particular embodiments, the one or more machine-learning algorithms may analyze a wide variety of data, allowing for detection of early cancer (e.g., Stage I, Stage II) and/or other diseases of interest.

FIG. 1B illustrates an example diagram 130 of example surface chemistry 122 and capture approaches. In particular embodiments, EV 102 may contain one or more biomarkers 104 on the surface of EV 102. In particular embodiments, label 140 may comprise, for example, an antibody, wherein label 140 may bind to a biomarker 104 on EV 102 and wherein label 140 may be captured by an antibody 120 on the surface As another example and not by way of limitation, label 140 may comprise an antibody and an MNP, wherein a capture antibody 120 may bind to the label antibody, resulting in an MNP proximity event. In particular embodiments, as demonstrated in FIG. 1B, diagram 130 may represent configuration “B” of Table 1, wherein label 140 may label an antibody, there is no label release mechanism, the surface capture mechanism is a secondary (capture) antibody binding to the EV label antibody, and the event detected at binding event 110 is antibody-antibody binding.

In particular embodiments, as demonstrated in FIG. 1B, diagram 130 may represent configuration “D” of Table 1. As an example, and not by way of limitation, label 140 may label an antibody-MNP without a label release mechanism, the surface capture mechanism may be secondary capture antibody binding to the EV label antibody 140, and the event detected at binding event 110 may be an MNP proximity event.

In particular embodiments, the event 110 may be detected by working electrode surface 124 and surface chemistry 122.

FIG. 1C illustrates an example diagram 150 of example surface chemistry 122 and capture approaches. In particular embodiments EV 102 may contain one or more biomarkers 104 and one or more labels 140 on the surface of EV 102. In particular embodiments, as represented in FIG. 1C, diagram 150 may represent configuration “C” of Table 1, wherein label 140 may comprise an antibody-enzyme conjugate without a label release mechanism, the surface capture mechanism may be a secondary antibody 120 binding to the EV label antibody, and the event detected at event 160 may be enzymatic activity.

In particular embodiments, the event 160 may be detected by working electrode surface 124 and surface chemistry 122.

FIG. 1D illustrates an example diagram 170 of example surface chemistry 122 and capture approaches. In particular embodiments, EV 102 may contain one or more biomarkers 104 and one or more labels 140 on the surface of EV 102. In particular embodiments, as represented in FIG. 1D, diagram 170 may represent configuration “E-1” of Table 1, wherein label 140 may be an antibody-ssDNA barcode with a chemical label release mechanism. As an example, and not by way of limitation, the label release mechanism may be represented by step 180, wherein the label may be cleaved from EV 102. In this example, biomarker 104 and label 140 may be measured in the absence of EV 102. As an example, and not by way of limitation, label 140 may represent an antibody-DNA and/or an antibody-DNA-MNP combination. In the example of configuration “E-1”, the surface capture mechanism may be a DNA barcode hybridizing to complementary ssDNA, wherein the detected binding event 110 may be DNA hybridization.

In particular embodiments, as represented in FIG. 1D, diagram 170 may represent configuration “E-2” of Table 1, wherein label 140 may be an antibody-ssDNA barcode with a photo label release mechanism. In the example of configuration “E-2”, the surface capture mechanism may be a DNA barcode hybridizing to complementary ssDNA, wherein the detected binding event 110 may be DNA hybridization.

In particular embodiments, as represented in FIG. 1D, diagram 170 may represent configuration “E-3” of Table 1, wherein label 140 may be an antibody-ssDNA barcode with an electrochemical label release mechanism. In the example of configuration “E-3”, the surface capture mechanism may be a DNA barcode hybridizing to complementary ssDNA, wherein the detected binding event 110 may be DNA hybridization.

In particular embodiments, diagram 170 may represent configuration “G-1” of Table 1, wherein label 140 may be an antibody-ssDNA barcode-MNP with a chemical label release mechanism. In the example of configuration “G-1”, the surface capture mechanism may be a DNA barcode hybridizing to complementary ssDNA, wherein the detected binding event 110 may be MNP proximity.

In particular embodiments, diagram 170 may represent configuration “G-2” of Table 1, wherein label 140 may be an antibody-ssDNA barcode-MNP with a photo label release mechanism. In the example of configuration “G-2”, the surface capture mechanism may be a DNA barcode hybridizing to complementary ssDNA, wherein the detected binding event 110 may be MNP proximity.

In particular embodiments, diagram 170 may represent configuration “G-3” of Table 1, wherein label 140 may be an antibody-ssDNA barcode-MNP with an electrochemical label release mechanism. In the example of configuration “G-3”, the surface capture mechanism may be a DNA barcode hybridizing to complementary ssDNA, wherein the detected binding event 110 may be MNP proximity.

In particular embodiments, the event 110 may be detected by working electrode surface 124 and surface chemistry 122.

FIG. 1E illustrates an example diagram 190 of example surface chemistry 122 and capture approaches. In particular embodiments, EV 102 may contain one or more biomarkers 104 and one or more labels 140 on the surface of EV 102. In particular embodiments, as represented in FIG. 1E, diagram 190 may represent configuration “F-1” of Table 1, wherein the EV label may be an antibody-ssDNA barcode with a chemical label release mechanism. As an example, and not by way of limitation, the label release mechanism may be represented by step 180, wherein the label may be cleaved from EV 102. In this example, label 140 may be measured in the absence of EV 102. As an example, and not by way of limitation, label 140 may represent an antibody-DNA combination and/or an antibody-DNA-enzyme combination. In the example of configuration “F-1”, the surface capture mechanism may be a DNA barcode hybridizing to complementary ssDNA, wherein the detected event 160 may be polymerase activity using a DNA polymerase.

In particular embodiments, diagram 190 may represent configuration “F-2” of Table 1, wherein label 140 may be an antibody-ssDNA barcode with a photo label release mechanism. In the example of configuration “F-2”, the surface capture mechanism may be a DNA barcode hybridizing to complementary ssDNA, wherein the detected event 160 may be polymerase activity using a DNA polymerase.

In particular embodiments, diagram 190 may represent configuration “F-3” of Table 1, wherein label 140 may be an antibody-ssDNA barcode with an electrochemical label release. In the example of configuration “F-3”, the surface capture mechanism may be a DNA barcode hybridizing to complementary ssDNA, wherein the detected event 160 may be polymerase activity using a DNA polymerase.

In particular embodiments, diagram 190 may represent configuration “H-1” of Table 1, wherein label 140 may be an antibody-ssDNA barcode-enzyme with a chemical label release mechanism. In the example configuration “H-1”, the surface capture mechanism may be DNA barcode hybridizing to complementary ssDNA, wherein the detected event 160 may be polymerase activity using a DNA polymerase.

In particular embodiments, diagram 190 may represent configuration “H-2” of Table 1, wherein label 140 may be an antibody-ssDNA barcode-enzyme with a photo label release mechanism. In the example configuration “H-2”, the surface capture mechanism may be DNA barcode hybridizing to complementary ssDNA, wherein the detected event 160 may be polymerase activity using a DNA polymerase.

In particular embodiments, diagram 190 may represent configuration “H-3” of Table 1, wherein label 140 may be an antibody-ssDNA barcode-enzyme with an electrical label release mechanism. In the example configuration “H-3”, the surface capture mechanism may be DNA barcode hybridizing to complementary ssDNA, wherein the detected event 160 may be polymerase activity using a DNA polymerase.

In particular embodiments, the event 160 may be detected by working electrode surface 124 and surface chemistry 122.

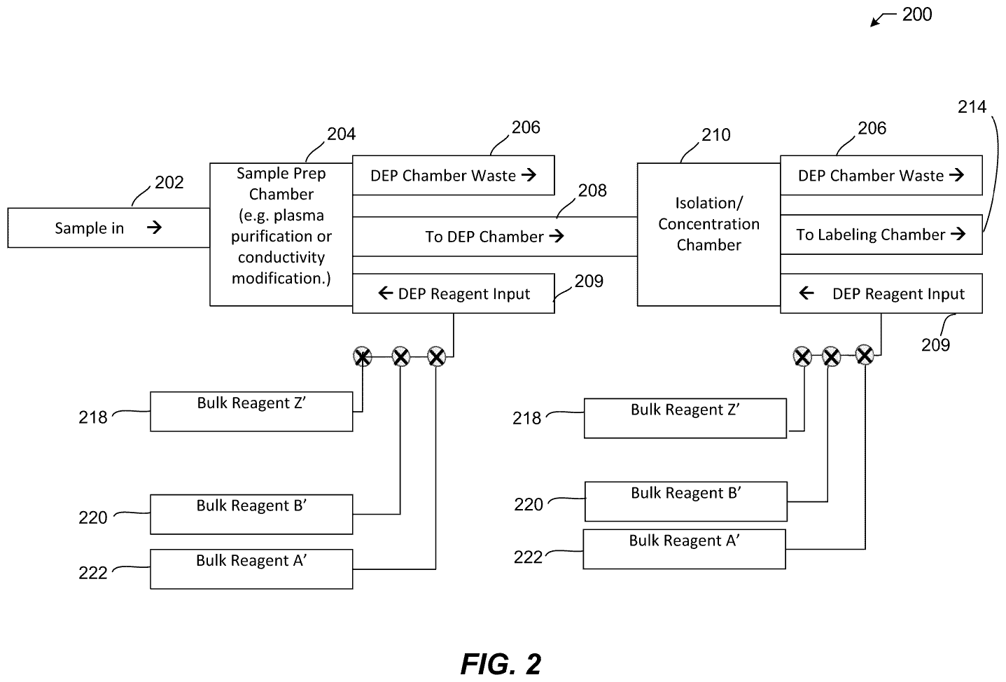

FIG. 2 illustrates an example flowchart 200 of sample preparation and isolation and/or concentration chambers. In particular embodiments, flowchart 200 of FIG. 2 may describe chamber and fluid channel architecture for sample preparation and isolation/concentration chambers. In particular embodiments, flowchart 200 may begin at step 202 where the biological sample is placed within the system and passed to the sample preparation chamber 204. As an example, and not by way of limitation, the sample preparation chamber may conduct a plasma purification process. As another example and not by way of limitation, the sample preparation chamber may conduct conductivity modification. As an example, and not by way of limitation, isolation and/or concentration may be achieved using DEP.

In particular embodiments, flowchart 200 may continue by passing the biological sample to the DEP chamber 208, wherein DEP chamber waste 206 may be output from sample prep chamber 204 and DEP reagent input 209 may be input to sample preparation chamber 204.

In particular embodiments, the biological sample may be passed from sample preparation chamber 204 to the isolation/concentration chamber 210. In particular embodiments, multiple fluid reagents may be supplied to and from one or more fluid chambers using bulk and/or consumable reagent containers. In particular embodiments, the multiple fluid reagents may be supplied to and from one or more fluid chambers using a fluid directing manifold and a motive force that is either hydraulic, pneumatic, or electrostatic in nature. In the example where electrostatic forces are used, a method of electrowetting the fluids that use electrodes may be used, wherein the electrodes may be located on either the top or bottom of the fluid channels and chambers and wherein the electrodes may be coated with thin films selected to achieve a hydrophobicity or hydrophilicity to support electrowetting.

In particular embodiments, DEP chamber waste 206 may be output from the isolation/concentration chamber 210 and DEP reagent input 209 may be input to the isolation/concentration chamber 210. In particular embodiments, DEP reagent input 209 may consist of one or more bulk reagents, such as bulk reagent Z′ 218, bulk reagent B′ 220, and/or bulk reagent A′.

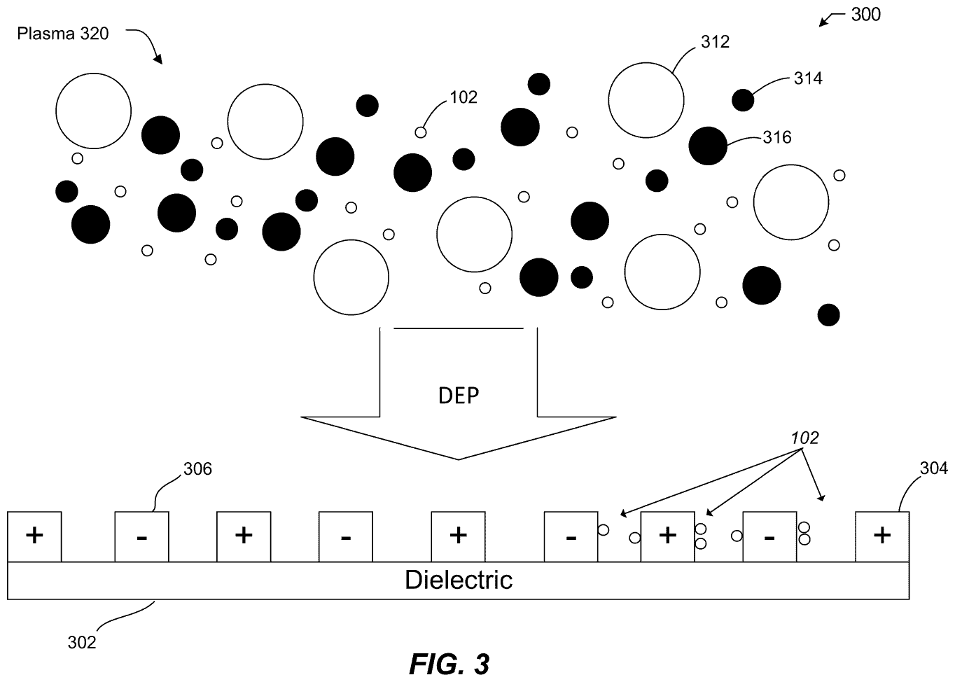

FIG. 3 illustrates an example diagram 300 of a cross section view of a dielectrophoresis concept. In particular embodiments, the isolation of one or more EVs 102 may be accomplished in the detection apparatus by using DEP, wherein DEP is the net force that acts on particles with an asymmetric polarizability in the presence of a radio frequency (RF) field. As demonstrated by diagram 300, plasma 320 of the biological sample may enter a DEP chamber (e.g., isolation and/or concentration chamber 210). As an example, and not by way of limitation, plasma 320 may consist of EVs 102 as well as various other biological particles 312, 314, 316. In this example, EVs 102 may be measured as spherical objects of roughly 30-150 nm. In particular embodiments, when an alternating current (AC) waveform is run through positive electrodes 304 and negative electrodes 306, the one or more EVs 102 may experience an attractive force that is a function of the RF frequency, voltage, plasma conductivity, EV particle size, and EV particle charge. In this example, the positive electrodes 304 and/or negative electrodes 306 may be constructed with metal or any other suitable material. In particular embodiments, the positive electrodes 304 and/or negative electrodes 306 may be separated by dielectric material 302. In particular embodiments, EVs 102 may be attracted to one or more of the positive electrodes 304 and/or negative electrodes 306 on the surface of the dielectric material 302.

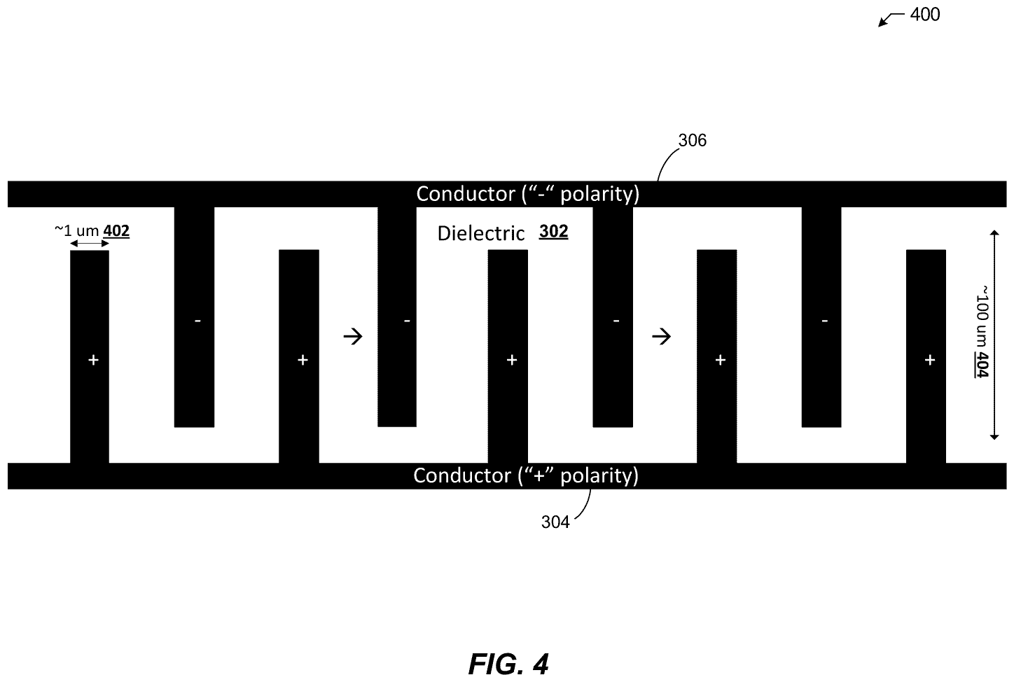

FIG. 4 illustrates an example diagram 400 of a dielectrophoresis electrode array. In particular embodiments, the one or more positive electrodes 304 and/or one or more negative electrodes 306 may be arranged in an interdigitated configuration. As an example, and not by way of limitation, the interdigitated configuration may create one or more regions with steep electric field gradients. In this example, the length of the electrodes 404 may be measured in hundreds (100s) of microns to millimeters, wherein the width of the electrodes 402 may be under one (1) micron. Similarly, the pitch and thickness of the electrodes may be under one (1) micron.

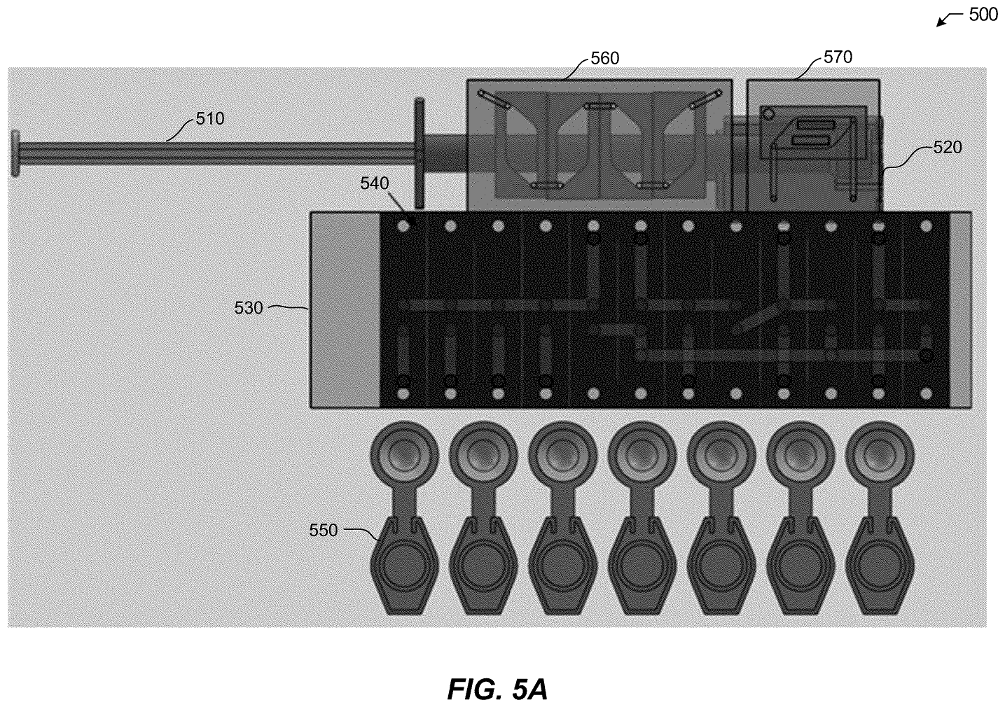

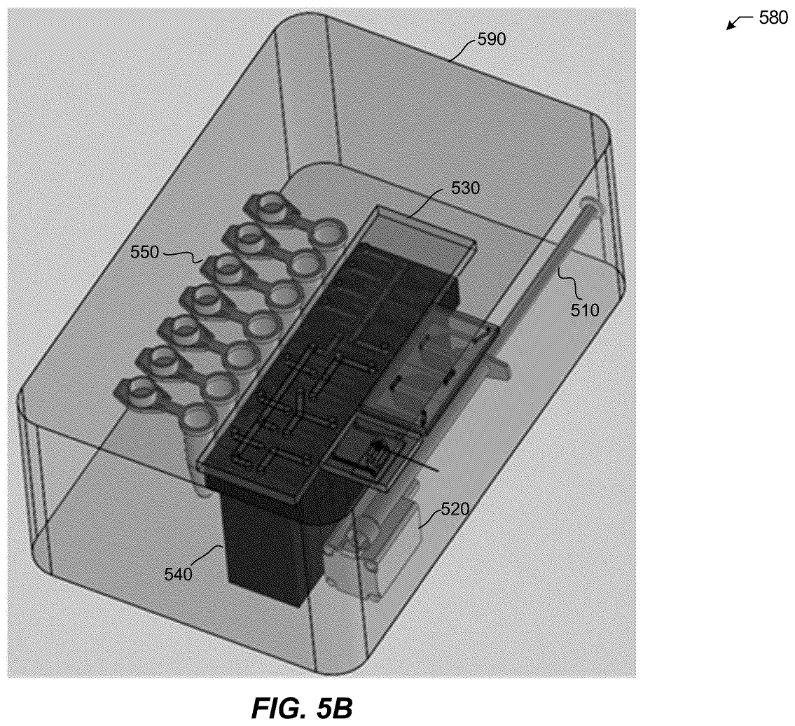

FIG. 5A illustrates an example isometric diagram 500 of a generalized fluidics delivery approach of a detection apparatus. In particular embodiments, the detection apparatus may be a consumable chip (e.g., printed circuit board), wherein one or more biological samples (e.g., fluid) may be injected into the detection apparatus by one or more syringes 510.

In particular embodiments, one or more syringes 510 of the detection apparatus may be actuated by one or more motors 520, wherein motor 520 may contain one or more gears. In particular embodiments, the detection apparatus may move fluid (e.g., a biological sample) through manifold 530 to a connected plurality of solenoid actuated values 540. It is understood that the solenoid actuated valves may be arranged in parallel, series, or any other suitable configuration.

In particular embodiments, the detection apparatus may move fluid through one or more reservoirs 550, wherein the one or more reservoirs 550 may be constructed by centrifuge tubes or any other suitable material. In particular embodiments, one or more syringes 510 of the detection apparatus may be actuated by one or more motors 520, wherein motor 520 may contain one or more gears. In particular embodiments, the detection apparatus may move fluid (e.g., a biological sample) through manifold 530 to a connected plurality of solenoid actuated values 540. It is understood that the solenoid actuated valves may be arranged in parallel, series, or any other suitable configuration. In particular embodiments, the detection apparatus may move fluid through one or more reservoirs 550, wherein the one or more reservoirs 550 may be constructed by centrifuge tubes or any other suitable material.

In particular embodiments, the detection apparatus may move fluid through one or more isolation and/or tagging chambers 560, wherein tagging of the fluid and/or isolation of the fluid may occur. As an example, and not by way of limitation, the one or more isolation and/or tagging chambers 560 may tag particular chemical groups.

In particular embodiments, the detection apparatus may move fluid through and one or more sensor chambers 570. As an example, and not by way of limitation, the detection apparatus may receive one or more biological samples via one or more reservoirs 550, wherein the biological sample may be passed to one or more tagging and/or isolation chambers 560 and subsequently passed to one or more sensor chambers 570. Although this disclosure discusses a particular order of processing biological samples within the detection apparatus, this disclosure contemplates any suitable order of processing biological samples within the detection apparatus.

In particular embodiments, particular bulk reagents may be dedicated to one or more specific chambers 560 As an example and not by way of limitation, chambers 560 may include detector chambers, wherein the detector chambers may receive one or more “detector” reagents. As another example and not by way of limitation, particular detector reagents may be input to one or more particular chambers 560, or a particular detector reagent may be common to all of chambers 560. As an example, and not by way of limitation, each chamber of the one or more chambers 560 may label a particular biomarker. For example, one chamber 560 may label one particular biomarker, such as “biomarker 1,” wherein another chamber 560 may label “biomarker 2.” In this example, one or more chambers 560 may cleave specific labels, wherein the labels may be chemically bonded to one or more sensors and ultimately digitally quantified. In particular embodiments, the one or more chambers 560 may receive fluid (e.g., biological sample) input and output waste from each particular chamber.

FIG. 5B illustrates an example isometric diagram 580 of a generalized fluidics delivery approach. In particular embodiments, the assembly of the detection apparatus as discussed in diagram 500 of FIG. 5A may be enclosed within box 590. As an example and not by way of limitation, box 590 may house the one or more syringes 510, one or more motors 520, manifold 530, one or more solenoid-actuated valves 540, one or more reservoirs 550, one or more chambers 560, and one or more sensors 570.

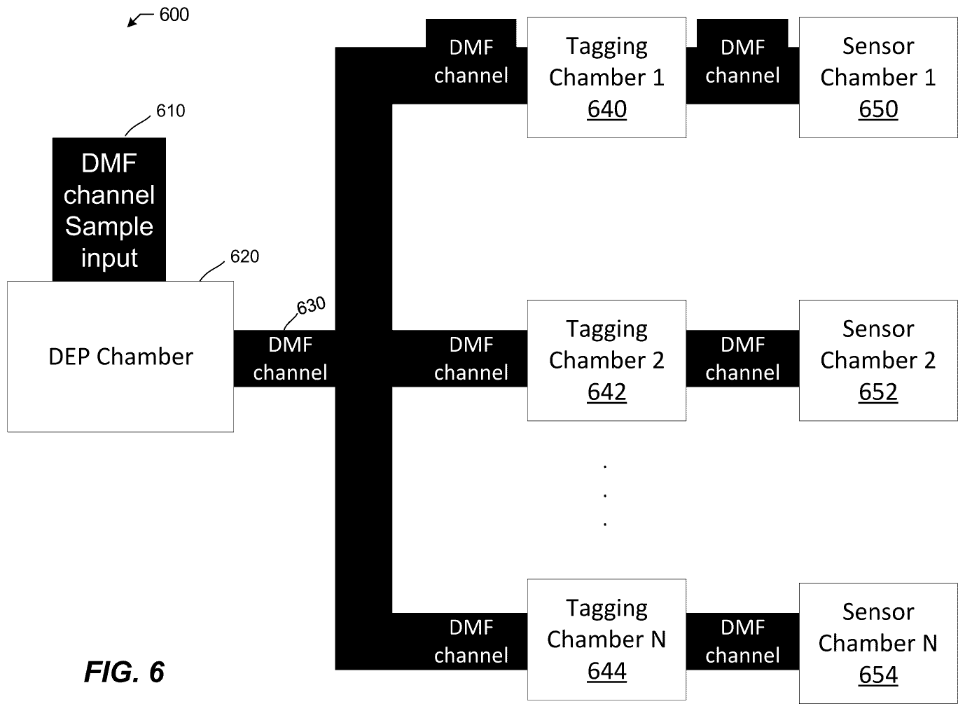

FIG. 6 illustrates an example diagram 600 of device architecture for interrogating multiple EV-derived biomarkers 104 from one or more biological samples using fluidic multiplexing. In particular embodiments, isolation of a biological sample may be performed in a single chamber, wherein sample sub-volumes of the biological sample may be routed to biomarker-specific labeling and detection chambers (e.g., chambers 560). As an example, and not by way of limitation, one or more reservoirs 550 containing biological samples may be input to the device via digital microfluidics sample input 610, wherein the one or more biological samples may be routed to dielectrophoresis (DEP) chamber 620. It is understood that DEP chamber 620 may be the only chamber within the device of diagram 600, or DEP chamber 620 may be one of many DEP chambers 620 within the device of diagram 600.

In particular embodiments, DEP chamber 620 may process the one or more biological samples, wherein biological sample may be passed to digital microfluidics (DMF) channel 630 until biological sample reaches one or more of a tagging chamber. It is understood that DMF channel 630 may also be a microfluidics channel as opposed to a digital microfluidics (DMF) channel. As displayed by diagram 600 of FIG. 6, the one or more biological samples may be processed and tagged in a plurality of chambers, such as tagging chamber “1” 640, tagging chamber “2” 642, and/or tagging chamber “N” 644. In particular embodiments, biological samples may be passed from the one or more tagging chambers 640, 642, 644 to one or more sensor chambers 650, 652, and/or 654 via DMF channel 630. In particular embodiments, a single, common detector chemistry may be used for each of the sensor chambers 650, 652, and/or 654. In this example, the chemistry may be common, so the specificity of what is detected may be determined by the label, wherein after the one or more biological samples pass through tagging chambers 640, 642, and/or 644, only labeled exosomes of biological samples may be detected. In this example, each of the N labeling chambers may label one distinct and specific biomarker only. In particular embodiments, in one or more tagging chambers (e.g., tagging chambers 640, 642, 644), EV and/or biomarker specific labels may be cleaved from EV 102 and subsequently chemically bonded to one or more sensors (e.g., sensor 570), wherein the labels may be digitally quantified or quantified through suitable means.

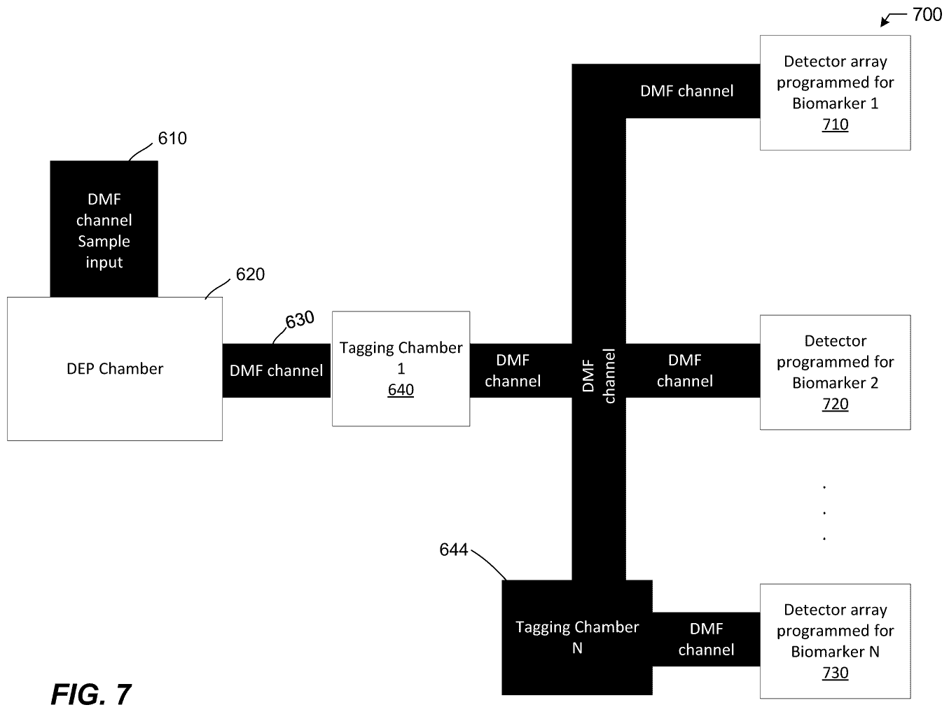

FIG. 7 illustrates an example diagram 700 of device architecture for interrogating multiple EV-derived biomarkers 104 from one or more biological samples using fluidic multiplexing. In particular embodiments, isolation of one or more biological sample may be performed in a single chamber, wherein sub-volumes of the one or more biological samples may be routed to biomarker-specific labeling and detection chambers. As an example, and not by way of limitation, one or more biological samples may be input to the device via DMF sample input 610, wherein the one or more biological samples may be routed to DEP chamber 620. It is understood that DEP chamber 620 may be the only chamber within the device of diagram 700, or DEP chamber 620 may be one of many DEP chambers 620 within the device of diagram 700.

In particular embodiments, DEP chamber 620 may process the one or more biological samples, wherein a biological sample may be passed to digital microfluidics (DMF) channel 630 until the biological sample reaches one or more of a tagging chamber. As displayed by diagram 700 of FIG. 7, the one or more biological samples may be processed and tagged in a plurality of chambers, such as tagging chamber “1” 640, and/or tagging chamber “N” 644. In particular embodiments, the one or more biological samples may be passed from the one or more tagging chambers 640, 644 to one or more detector arrays 710, 720, 730 via DMF channel 630. In particular embodiments, each of a plurality of detector arrays may be programmed to detect a particular biomarker. As an example, and not by way of limitation, detector array 710 may be specifically programmed for a particular biomarker, such as “biomarker 1.” Similarly, detector array 720 may be programmed for “biomarker 2” and detector array 730 may be programmed for “biomarker N.” In this example, each detector array may be programmed to operate with a different detector chemistry, wherein each detector chamber may detect attachment of one distinct and specific labeled biomarker. In particular embodiments, EV and/or biomarker specific labels may be cleaved from EVs 102 in tagging chamber “1” 640, wherein the label may be chemically bonded to one or more sensors (e.g., detector arrays 710, 720, 730) and digitally quantified.

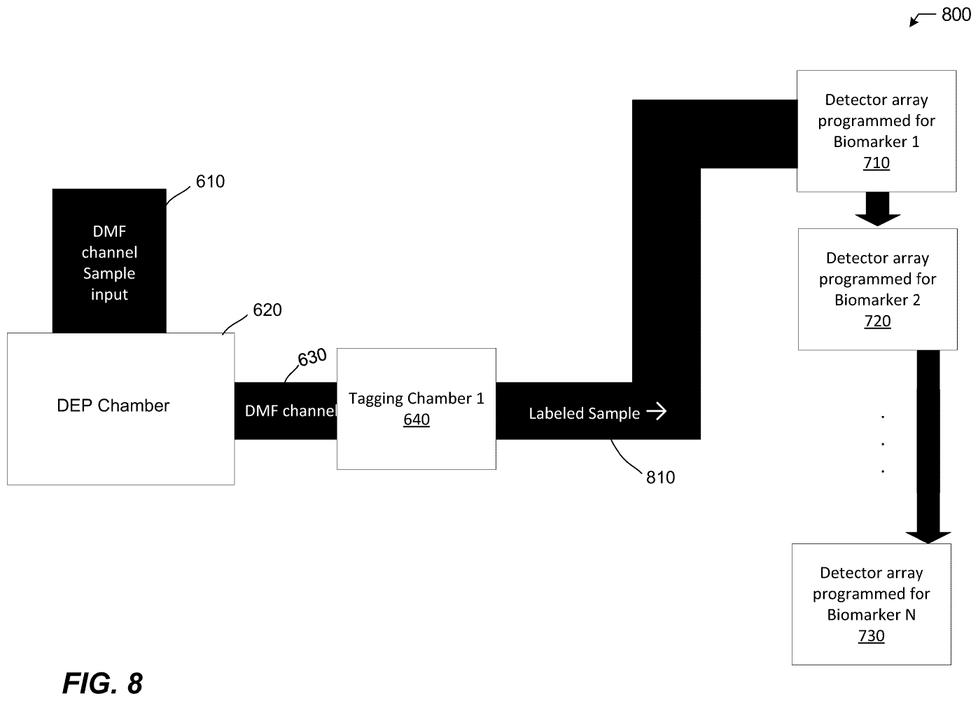

FIG. 8 illustrates an example diagram 800 of device architecture for interrogating multiple EV-derived biomarkers 104 from one or more biological samples using fluidic multiplexing. In particular embodiments, isolation of one or more biological samples may be performed in a single chamber, wherein sub-volumes of the one or more biological samples may be routed to biomarker-specific labeling and detection chambers. As an example, and not by way of limitation, one or more biological samples may be input to the device via DMF sample input 610, wherein the one or more biological samples may be routed to DEP chamber 620. It is understood that DEP chamber 620 may be the only chamber within the device of diagram 800, or DEP chamber 620 may be one of many DEP chambers 620 within the device of diagram 800.

In particular embodiments, DEP chamber 620 may process the one or more biological samples, wherein the biological sample(s) may be passed to digital microfluidics (DMF) channel 630 until the biological sample(s) reach one or more of a tagging chamber. As an example, and not by way of limitation, DMF channel 630 may be a microfluidics channel. As displayed by diagram 800 of FIG. 8, the one or more biological samples may be processed and tagged by a singular tagging chamber, such as tagging chamber “1” 640, wherein the biological sample may be tagged and labeled. As an example, and not by way of limitation, the labeled sample 810 may be passed from tagging chamber “1” 640 to one or more detector arrays 710, 720, 730 via DMF Channel 630 or a microfluidics channel. As discussed in FIG. 7, each of a plurality of detector arrays may be programmed to detect a particular biomarker. As an example, and not by way of limitation, detector array 710 may be specifically programmed for a particular biomarker, such as “biomarker 1.” Similarly, detector array 720 may be programmed for “biomarker 2” and detector array 730 may be programmed for “biomarker N.” In the example of diagram 800 of FIG. 8, the volume of labeled EVs (e.g., labeled sample 810) may be passed through all detection chambers (e.g., detector arrays 710, 720, 730), thereby increasing efficiency in biological sample usage. As discussed in FIGS. 6-7, EV and/or biomarker specific labels may be cleaved from EVs 102 in tagging chamber “1” 640 and subsequently chemically bonded to one or more sensor, wherein the labels may be digitally quantified.

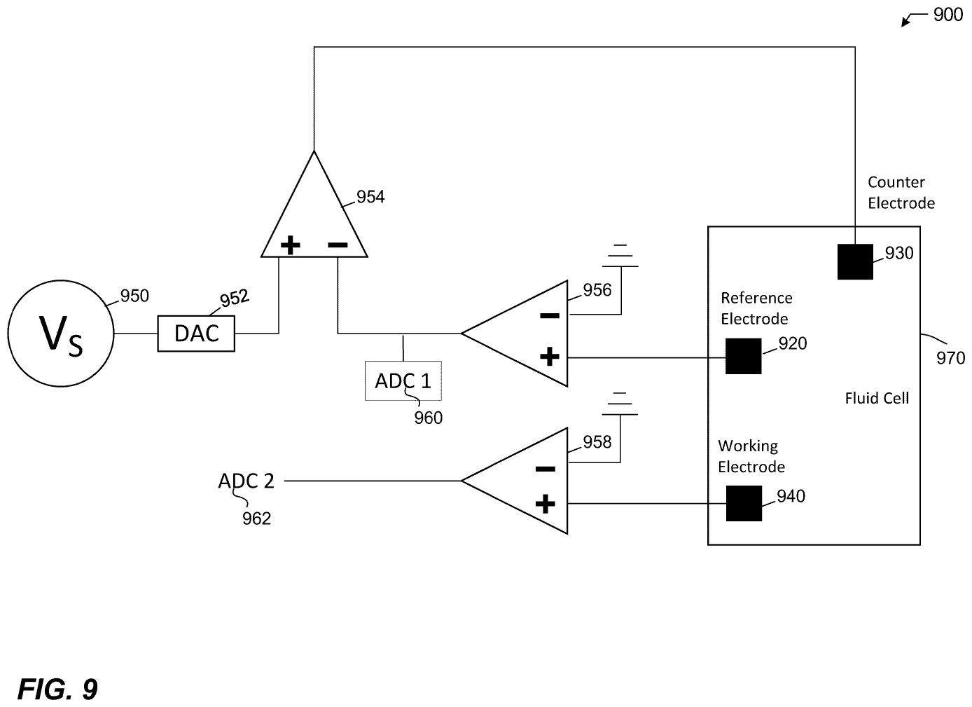

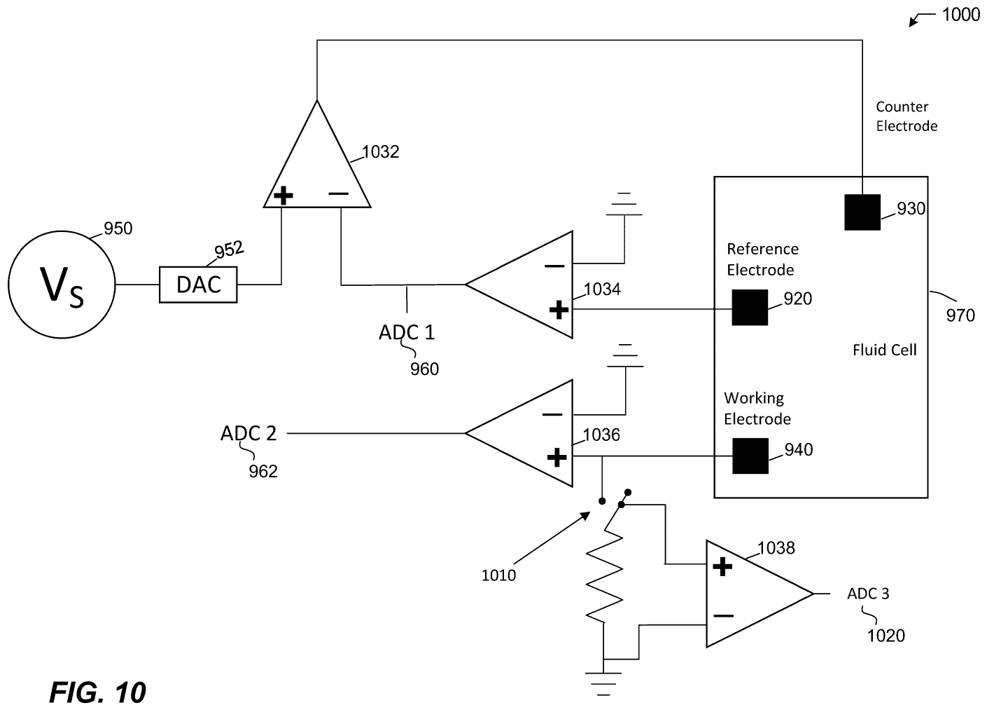

FIG. 9 illustrates an example schematic diagram 900 displaying open circuit potential of a working electrode. In particular embodiments, the circuit architecture of a device displayed by diagram 900 may be used to measure the change in floating voltage of working electrode 940 compared to reference electrode 920 held at potential by counter electrode 930. As an example, and not by way of limitation, the circuit of diagram 900 may include voltage source 950, digital-to-analog converter (DAC) 952, analog-to-digital converters 960, 962, diodes 954, 956, 958, reference electrode 920, counter electrode 930, and working electrode 940. In particular embodiments, a method of preparing an electronic circuit to detect one or more events as previously discussed may consist of an independent reference electrode (e.g., reference electrode 920), counter electrode 930, and working electrode 940 to detect immunochemical potential changes, immunochemical impedance changes, and/or immunochemical current changes using either an open circuit potential configuration, an electrochemical impedance spectroscopy configuration, a cyclic voltammetry configuration, or an ammeter configuration (as displayed in diagram 900 of FIG. 9 and diagram 1000 of FIG. 10). As an example and not by way of limitation, reference electrode 920, counter electrode 930, and/or working electrode 940 may be fabricated from any metal, conductive metal oxide, or suitable conductive material (e.g., graphene) including but not limited to Pt, Au, Ag, Ag/AgCl, Zn, Ti, W, Pd, Ru, Pb, Cu, In, InxOy, ITO, AZO, ICO, graphene, etc.

In particular embodiments, each of reference electrode 920, counter electrode 930, and/or working electrode 940 may be fabricated from distinct materials. In particular embodiments, any one of reference electrode 920, counter electrode 930, and/or working electrode 940 may be functionalized, while the remaining electrodes are not functionalized. As an example, and not by way of limitation, working electrode 940 may be functionalized while reference electrode 920 and counter electrode 930 may not be functionalized. In particular embodiments, the potential, impedance, and/or current at each of reference electrode 920, counter electrode 930, and/or working electrode 940 may be monitored with one or more ADCs (e.g., ADCs 960, 962), wherein the current may be stored for determination of biomarker quantification.

FIG. 10 illustrates an example schematic diagram 1000 displaying an example electrode configuration. In particular embodiments, the circuit architecture of a device displayed by diagram 1000 may be used to measure the change in floating voltage of working electrode 940 compared to reference electrode 920 held at potential by counter electrode 930. As an example, and not by way of limitation, the circuit of diagram 1000 may include voltage source 950, digital-to-analog converter (DAC) 952, analog-to-digital converters 960, 962, 1020, operational amplifiers 1032, 1034, 1036, 1038, reference electrode 920, counter electrode 930, and working electrode 940. In particular embodiments, switch 1010 may be added to the open circuit potential, which may allow for amperometric measurements and/or electrochemical impedance spectroscopy (EIS).

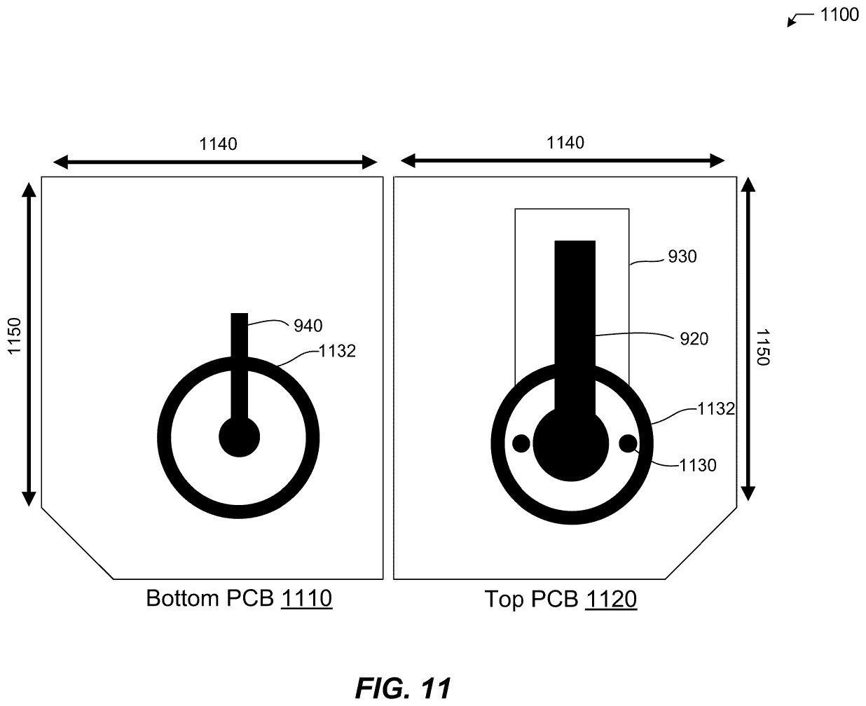

FIG. 11 illustrates an example diagram 1100 of an electrode configuration in which one embodiment may operate. In particular embodiments, bottom printed circuit board (PCB) 1110 may consist of a gasket 1132 and working electrode 940. As an example, and not by way of limitation, length 1150 of bottom PCB 1110 may measure approximately 12.7 millimeters, and width 1140 of bottom PCB 1110 may measure approximately 12.7 millimeters. In particular embodiments, top PCB 1120 may consist of gasket 1132, one or more vents 1130, reference electrode 920, and counter electrode 1130. As an example, and not by way of limitation, length 1150 of top PCB 1120 may measure approximately 12.7 millimeters, and width 1140 of top PCB 1120 may measure approximately 12.7 millimeters.

In particular embodiments, the detection apparatus (e.g., device) for quantifying EV-derived biomarkers in a biological sample may include a device platform capable of holding a cartridge. In particular embodiments, the detection apparatus (e.g., device) for quantifying EV-derived biomarkers in one or more biological samples (e.g., blood, plasma, serum, cerebrospinal fluid, lymphatic fluid, saliva, urine, fecal matter, cell lysate, cell culture fluid) may include a device platform capable of holding a cartridge and delivering samples, reagents, light, and/or electrical pulses to multiple fluid and electrical channels on a cartridge. As an example, and not by way of limitation, the device platform may include an electronics board with logic and/or power circuitry for driving and sensing electronic devices, sensors, and/or electrodes. As another example and not by way of limitation, the device platform may include a fluidics control system consisting of fluid reservoirs, tubing, and either pneumatic, hydraulic, or electrical fluidical controls, wherein the fluidic controls may be manually and/or electronically actuated.

In particular embodiments, a fluidics cartridge may be constructed by a molded top material and molded bottom material, wherein a space between the top and bottom molded material may house one or more printed circuit boards and/or microelectronics chip(s). As an example, and not by way of limitation, the top molded material and bottom molded material may be constructed of plastic or any other suitable material. In particular embodiments, one or more adhesive layer(s) may create a fluidic seal between chambers and/or channels of the fluidics cartridge. In particular embodiments, a “spacer” layer in conjunction with the one or more printed circuit board(s) may define the boundaries of the fluid chambers and/or channels. As an example, and not by way of limitation, the spacer may be constructed of plastic or any other suitable material. In particular embodiments, the fluidics cartridge may include ports dedicated to the ingress and egress of fluids for each channel. In particular embodiments, components comprising the walls of one or more fluid chambers and/or channels may be coated with a non-biofouling film.

In particular embodiments, the detection apparatus for quantifying EV-derived biomarkers in a biological sample may include a fluidics cartridge with multiple chambers and/or channels. In particular embodiments, a detection apparatus for quantifying EV-derived biomarkers in a biological sample may include printed circuits and/or a microelectronics silicon-based integrated chip(s) (e.g., complementary metal-oxide semiconductor (CMOS) or silicon-based integrated chip), wherein the chip(s) may be constructed to create a DEP cavity (e.g., DEP chamber 620). As an example, and not by way of limitation, the DEP cavity may isolate and capture particles of a tunable size and/or charge range from one or more biological samples (e.g., plasma sample).

In particular embodiments, the device for quantifying EV-derived biomarkers 104 in a plasma sample may include a printed circuit board and/or a silicon-based integrated chip with electrodes, wherein the printed circuit board and/or silicon based integrated chip may electrostatically control one or more fluidic motion systems. In particular embodiments, the device for quantifying EV-derived biomarkers in a plasma sample may include a fluid supply (e.g., sample input 610) and/or waste management system. In particular embodiments, the device for quantifying EV-derived biomarkers in a biological sample may include one or more reaction chambers. As an example, and not by way of limitation, the one or more reaction chambers may include a DEP structure, wherein the DEP structure may support the labeling and/or cleaving of EV-derived biomarker labels.

In particular embodiments, the device for quantifying EV-derived biomarkers in a biological sample may include one or more printed circuits and/or microelectronic chips containing electrodes for electrical capturing and detecting of labeled EVs and/or cleaved EV-biomarker labels. In particular embodiments, the device for quantifying EV-derived biomarkers 104 in a biological sample may include a plurality of electronics and/or sensors, wherein the electronics and/or sensors may report digital sensor responses quantifying the presence of biomarkers 104 and/or biomarker labels.

In particular embodiments, the EV-biosensor device may perform DEP particle filtering to separate EVs 102 from other biological components, wherein the EV-biosensor device may be constructed by one or more silicon chips. As an example, and not by way of limitation, the top of the one or more silicon chips may be constructed by a metal layer, wherein the metal layer may contain an interdigitated alternating polarity electrode arrangement. In particular embodiments, the EV-biosensor device may contain an RF AC waveform generator off-chip, wherein an RF AC waveform generator may apply one or more of a particular waveform across one or more electrodes (e.g., reference electrode 920, counter electrode 930, working electrode 940). In particular embodiments, the EV-biosensor device may include an anti-bio-fouling surface coating or other coating on electrode and/or non-electrode surfaces within DEP chamber 620.

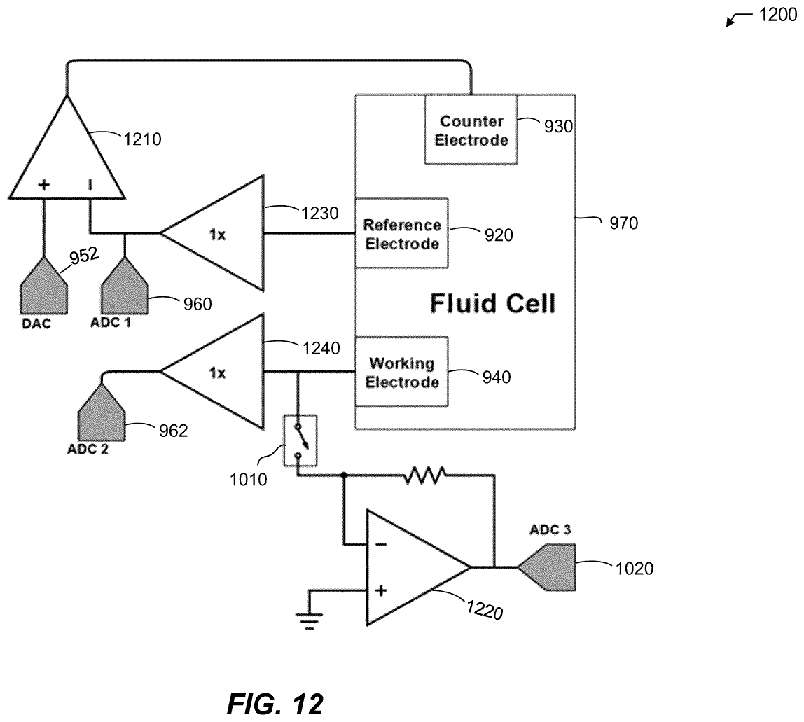

FIG. 12 illustrates an example schematic diagram 1200 displaying an example electrode configuration. In particular embodiments, the circuit architecture of a detection apparatus displayed by diagram 1200 with the switch in an “open” position may be used to measure the change in open circuit potential of the working electrode 940 as compared to reference electrode 920 held at potential by counter electrode 930. Alternatively, the circuit architecture of a device displayed by diagram 1200 with the switch in the “closed” position may be used to measure the current flowing through the working electrode 940 in response to a defined voltage on the reference electrode 920. As an example and not by way of limitation, the circuit of diagram 1300 may include digital-to-analog converters (DAC) 952, analog-to-digital converters (ADC) 960, 962, 1020, operational amplifiers 1210, 1220, buffers 1230, 1240, switch 1010, reference electrode 920, counter electrode 930, and working electrode 940. In particular embodiments, a method of preparing an electronic circuit to detect one or more events as previously discussed may consist of an independent reference electrode (e.g., reference electrode 920), counter electrode 930, and a working electrode 940 to detect immunochemical potential changes, immunochemical impedance changes, and/or immunochemical current changes using either an open circuit potential configuration, an electrochemical impedance spectroscopy configuration, a cyclic voltammetry configuration, or an ammeter configuration. As an example, and not by way of limitation, reference electrode 920, counter electrode 930, and/or working electrode 90 may be fabricated from any metal, conductive metal oxide, or suitable conductive material.

In particular embodiments, each of reference electrode 920, counter electrode 940, and/or working electrode 940 may be fabricated from distinct materials. In particular embodiments, any one of reference electrode 920, counter electrode 930, and/or working electrode 940 may be functionalized, while the remaining electrodes are not functionalized, or functionalized differently. In particular embodiments, the potential, impedance, and/or current at each of reference electrode 920, counter electrode 930, and/or working electrode 940 may be monitored with one or more ADCs (e.g., ADCs 960, 962, 1020) wherein the current may be stored for determination of biomarker quantification.

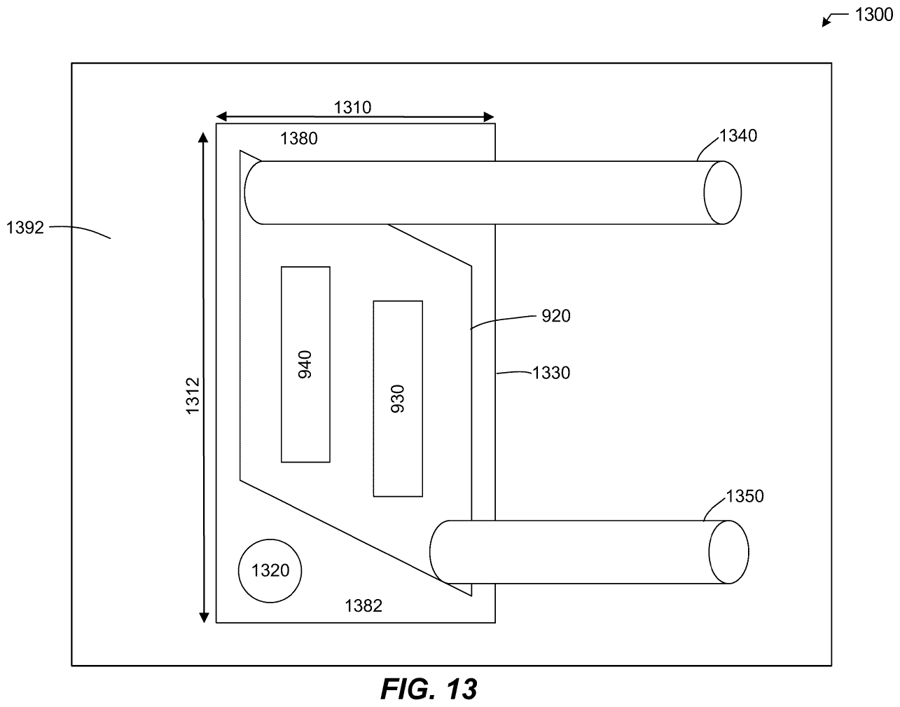

FIG. 13 illustrates an example diagram 1300 of an electrode configuration in which one embodiment may operate. In particular embodiments, bottom surface 1380 may consist of one or more working electrodes 940. As an example, and not by way of limitation, length 1312 of bottom surface 1380 may measure approximately twenty (20) millimeters. As another example and not by way of limitation, width 1310 of bottom surface 1380 may measure approximately ten (10) millimeters. In particular embodiments, top surface 1382 and bottom surface 1380 may be separated by one or more layers of transfer adhesive 1392 with one or more fluid ports 1340, 1350, and electrical pass-through 1320.

In particular embodiments, top surface 1382 may consist of electrical pass-through 1320, one or more reference electrodes 920, and one or more counter electrodes 930. As an example, and not by way of limitation, length 1312 of top surface 1382 may measure approximately twenty (20) millimeters and width 1310 of top surface 1382 may measure approximately ten (10) millimeters. Although this disclosure discusses an approximate length 1312 and width 1310 of top surface 1382 and bottom surface 1380, this disclosure contemplates any suitable length 1312 and width 1310. Although this disclosure discusses a particular electrode configuration (e.g., positioning of counter electrode 930, reference electrode 920, working electrode 940), this disclosure contemplates any suitable configuration of counter electrode 930, reference electrode 920, and/or working electrode 940.

### Systems and Methods

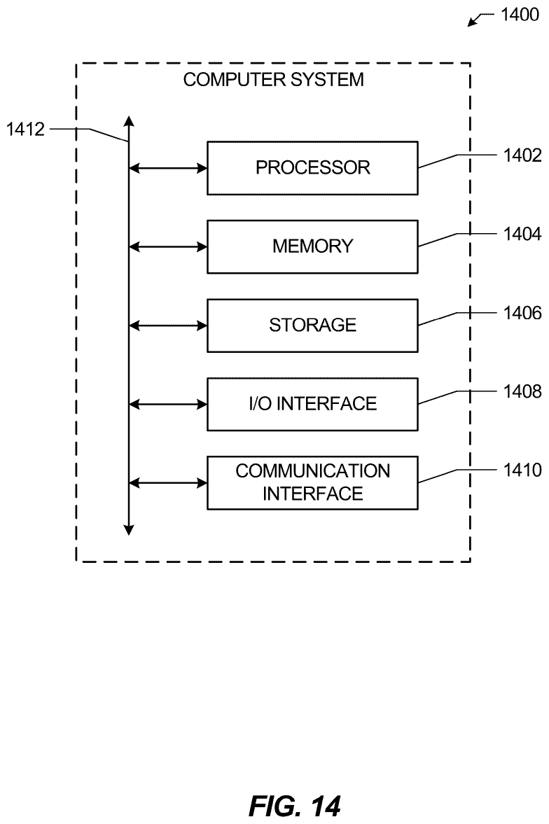

FIG. 14 illustrates an example computer system 1400 that may be utilized to perform digital, multiplexed, extracellular vesicle-derived biomarker lab-on-a-chip diagnostics, in accordance with the presently disclosed embodiments. In particular embodiments, one or more computer systems 1400 perform one or more steps of one or more methods described or illustrated herein. In particular embodiments, one or more computer systems 1400 provide functionality described or illustrated herein. In particular embodiments, software running on one or more computer systems 1400 performs one or more steps of one or more methods described or illustrated herein or provides functionality described or illustrated herein. Particular embodiments include one or more portions of one or more computer systems 1400. Herein, reference to a computer system may encompass a computing device, and vice versa, where appropriate. Moreover, reference to a computer system may encompass one or more computer systems, where appropriate.

This disclosure contemplates any suitable number of computer systems 1400. This disclosure contemplates computer system 1400 taking any suitable physical form. As example and not by way of limitation, computer system 1400 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 1400 may include one or more computer systems 1400; be unitary or distributed; span multiple locations; span multiple machines; span multiple data centers; or reside in a cloud, which may include one or more cloud components in one or more networks.

Where appropriate, one or more computer systems 1400 may perform without substantial spatial or temporal limitation one or more steps of one or more methods described or illustrated herein. As an example, and not by way of limitation, one or more computer systems 1400 may perform in real time or in batch mode one or more steps of one or more methods described or illustrated herein. One or more computer systems 1400 may perform at different times or at different locations one or more steps of one or more methods described or illustrated herein, where appropriate.

In particular embodiments, computer system 1400 includes a processor 1402, memory 1410, storage 1406, an input/output (I/O) interface 1408, a communication interface 1410, and a bus 1412. Although this disclosure describes and illustrates a particular computer system having a particular number of particular components in a particular arrangement, this disclosure contemplates any suitable computer system having any suitable number of any suitable components in any suitable arrangement. In particular embodiments, processor 1402 includes hardware for executing instructions, such as those making up a computer program. As an example, and not by way of limitation, to execute instructions, processor 1402 may retrieve (or fetch) the instructions from an internal register, an internal cache, memory 1410, or storage 1406; decode and execute them; and then write one or more results to an internal register, an internal cache, memory 1410, or storage 1406. In particular embodiments, processor 1402 may include one or more internal caches for data, instructions, or addresses. This disclosure contemplates processor 1402 including any suitable number of any suitable internal caches, where appropriate. As an example, and not by way of limitation, processor 1402 may include one or more instruction caches, one or more data caches, and one or more translation lookaside buffers (TLBs). Instructions in the instruction caches may be copies of instructions in memory 1410 or storage 1406, and the instruction caches may speed up retrieval of those instructions by processor 1402.

Data in the data caches may be copies of data in memory 1410 or storage 1406 for instructions executing at processor 1402 to operate on; the results of previous instructions executed at processor 1402 for access by subsequent instructions executing at processor 1402 or for writing to memory 1410 or storage 1406; or other suitable data. The data caches may speed up read or write operations by processor 1402. The TLBs may speed up virtual-address translation for processor 1402. In particular embodiments, processor 1402 may include one or more internal registers for data, instructions, or addresses. This disclosure contemplates processor 1402 including any suitable number of any suitable internal registers, where appropriate. Where appropriate, processor 1402 may include one or more arithmetic logic units (ALUs); be a multi-core processor; or include one or more processors 1402. Although this disclosure describes and illustrates a particular processor, this disclosure contemplates any suitable processor.

In particular embodiments, memory 1410 includes main memory for storing instructions for processor 1402 to execute or data for processor 1402 to operate on. As an example, and not by way of limitation, computer system 1400 may load instructions from storage 1406 or another source (such as, for example, another computer system 1400) to memory 1410. Processor 1402 may then load the instructions from memory 1410 to an internal register or internal cache. To execute the instructions, processor 1402 may retrieve the instructions from the internal register or internal cache and decode them. During or after execution of the instructions, processor 1402 may write one or more results (which may be intermediate or final results) to the internal register or internal cache. Processor 1402 may then write one or more of those results to memory 1410. In particular embodiments, processor 1402 executes only instructions in one or more internal registers or internal caches or in memory 1410 (as opposed to storage 1406 or elsewhere) and operates only on data in one or more internal registers or internal caches or in memory 140 (as opposed to storage 1406 or elsewhere).

One or more memory buses (which may each include an address bus and a data bus) may couple processor 1402 to memory 1410. Bus 1412 may include one or more memory buses, as described below. In particular embodiments, one or more memory management units (MMUs) reside between processor 1402 and memory 1410 and facilitate accesses to memory 1410 requested by processor 1402. In particular embodiments, memory 1410 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1410 may include one or more memory devices 1410, where appropriate. Although this disclosure describes and illustrates particular memory, this disclosure contemplates any suitable memory.

In particular embodiments, storage 1406 includes mass storage for data or instructions. As an example, and not by way of limitation, storage 1406 may include a hard disk drive (HDD), a floppy disk drive, flash memory, an optical disc, a magneto-optical disc, magnetic tape, or a Universal Serial Bus (USB) drive or a combination of two or more of these. Storage 1406 may include removable or non-removable (or fixed) media, where appropriate. Storage 1406 may be internal or external to computer system 1400, where appropriate. In particular embodiments, storage 1406 is non-volatile, solid-state memory. In particular embodiments, storage 1406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory, or a combination of two or more of these. This disclosure contemplates mass storage 1406 taking any suitable physical form. Storage 1406 may include one or more storage control units facilitating communication between processor 1402 and storage 1406, where appropriate. Where appropriate, storage 1406 may include one or more storages 1406. Although this disclosure describes and illustrates particular storage, this disclosure contemplates any suitable storage.

In particular embodiments, I/O interface 1408 includes hardware, software, or both, providing one or more interfaces for communication between computer system 1400 and one or more I/O devices. Computer system 1400 may include one or more of these I/O devices, where appropriate. One or more of these I/O devices may enable communication between a person and computer system 1400. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1406 for them. Where appropriate, I/O interface 1408 may include one or more device or software drivers enabling processor 1402 to drive one or more of these I/O devices. I/O interface 1408 may include one or more I/O interfaces 1406, where appropriate. Although this disclosure describes and illustrates a particular I/O interface, this disclosure contemplates any suitable I/O interface.

In particular embodiments, communication interface 1410 includes hardware, software, or both providing one or more interfaces for communication (such as, for example, packet-based communication) between computer system 1400 and one or more other computer systems 1400 or one or more networks. As an example, and not by way of limitation, communication interface 1410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1410 for it.

As an example, and not by way of limitation, computer system 1400 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1400 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1400 may include any suitable communication interface 1410 for any of these networks, where appropriate. Communication interface 1410 may include one or more communication interfaces 1410, where appropriate. Although this disclosure describes and illustrates a particular communication interface, this disclosure contemplates any suitable communication interface.

In particular embodiments, bus 1412 includes hardware, software, or both coupling components of computer system 1400 to each other. As an example, and not by way of limitation, bus 1412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1412 may include one or more buses 1412, where appropriate. Although this disclosure describes and illustrates a particular bus, this disclosure contemplates any suitable bus or interconnect.

### Miscellaneous

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A system for obtaining a diagnosis from a plurality of biomarkers, said system comprising:
means for receiving a biological sample onto an electrode array;
means for applying a dielectrophoretic force through a plurality of electrodes of said electrode array;
means for determining a quantity of biomarkers of interest in said biological sample.

2. The system of claim 1, wherein a strength and direction of dielectrophoretic force is specific to said biomarker of interest in said biological sample.

3. The system of claim 1, wherein a digital sensor determines said quantity of biomarkers of interest.

4. The system of claim 1, wherein said plurality of electrodes are interdigitated.

5. The system of claim 1, wherein said biomarker of interest is an extracellular vesicle.

6. The system of claim 1, wherein said biomarker is tagged with a label.

7. The system of claim 6, wherein said label is an antibody label, metal nanoparticle label, or single-stranded DNA label.

8. A method for obtaining a diagnosis from extracellular vesicle-derived biomarkers, said method comprising:
receiving a biological sample on an electrode array;
applying a dielectrophoretic force through a plurality of electrodes of said electrode array;
determining a quantity of biomarkers of interest in said biological sample.

9. The method of claim 8, wherein a strength and direction of dielectrophoretic force is specific to said biomarker of interest in said biological sample.

10. The method of claim 8, wherein a digital sensor determines said quantity of biomarkers of interest.

11. The method of claim 8, wherein said plurality of electrodes are interdigitated.

12. The method of claim 8, wherein said biomarker of interest is an extracellular vesicle.

13. The method of claim 8, wherein said biomarker is tagged with a label.

14. The method of claim 13, wherein said label is an antibody label, metal nanoparticle label, or single-stranded DNA label.

15. A system for obtaining a diagnosis from extracellular vesicle-derived biomarkers, said system comprising:
means for receiving a biological sample onto an electrode array;
means for applying a dielectrophoretic force through a plurality of electrodes of said electrode array;
means for filtering a first biomarker from a plurality of biomarkers of said biological sample;
means for tagging said first biomarker with a label;
means for detecting a quantity of said first biomarker; and
means for displaying a result.

16. The system of claim 15, wherein an electro-chemical sensor determines said quantity of biomarkers of interest.

17. The system of claim 15, wherein said plurality of electrodes are interdigitated.

18. The system of claim 15, wherein said biomarker of interest is an extracellular vesicle.

19. The system of claim 15, wherein said label is an antibody label, metal nanoparticle label, or single-stranded DNA label.

20. The system of claim 15, wherein said filtering is accomplished via a microfluidics channel.
